(19) United States
(12) Patent Application Publication
(10) Pub. No.: US 2010/0221784 A1
(43) Pub. Date: Sep. 2, 2010

(54) Beta-Glucosidase Enhanced Filamentous Fungal Whole Cellulase Compositions and Methods Of Use

(76) Inventors: Meredith K. Fujdala, San Jose, CA (US); Edmund A. Larenas, Moss Beach, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/676,333

(22) PCT Filed: Sep. 4, 2008
(86) PCT No.: PCT/US2008/010420

§ 371 Date: May 20, 2010

Related U.S. Application Data
(60) Provisional application No. 60/970,842, filed on Sep. 7, 2007.

(51) Int. Cl. C12P 19/00; C12N 9/42

(52) U.S. Cl. 435/72; 435/209

(57) ABSTRACT

The present disclosure provides beta-glucosidase enhanced filamentous fungal whole cellulase compositions. Also provided are methods of hydrolyzing a cellulosic material with beta-glu-cosidase enhanced whole cellulase compositions. The present disclosure further provides methods of decreasing the amount of a whole cellulase required to hydrolyze a cellulosic material by adding an effective amount beta-glucosidase.

### 1. CROSS-REFERENCE TO RELATED APPLICATIONS

This application claims the benefit of U.S. Provisional Application No. 60/970,842, filed on Sep. 7, 2007, which is incorporated by reference herein in its entirety.

### 2. FIELD

The present disclosure relates to the field of enzymes, and in particular, methods and compositions for the enzymatic hydrolysis of cellulosic materials.

### 3. INTRODUCTION

As the limits of non-renewable energy resources approach, the potential of cellulose as a renewable energy resource is enormous. Cellulose can be converted into sugars, such as glucose, and used as an energy source by numerous microorganisms including bacteria, yeast and fungi for industrial purposes. For example, cellulosic materials can be converted into sugars by enzymes, and the resulting sugars can be used as a feedstock for industrial microorganisms to produce products such as plastics and ethanol.

Utilization of cellulosic materials as a renewable carbon sources depends on the development of economically feasible cellulases for the enzymatic hydrolysis of cellulosic materials. Cellulases are enzymes which catalyze the hydrolysis of cellulose to products such as glucose, cellobiose, and other cellooligosaccharides. Cellulase enzymes work synergistically to hydrolyze cellulose to glucose. Exo-cellobiohydrolases (CBHs) such as CBHI and CBHII, generally act on the ends of cellulose to generate cellobiose, while the endoglucanases (EGs) act at random locations on the cellulose. Together these enzymes hydrolyze cellulose into smaller cello-oligosaccharides such as cellobiose. Cellobiose is hydrolyzed to glucose by beta-glucosidase.

Although many microorganisms are capable of degrading cellulose, only a few of these microorganisms produce significant quantities of enzymes capable of completely hydrolyzing crystalline cellulose. Accordingly, there remains a need to develop efficient enzyme systems for hydrolyzing cellulose for industrial applications. It is therefore desired to improve the efficiency and economics of the enzymatic hydrolysis of cellulosic materials.

### 4. SUMMARY

The present teachings provide beta-glucosidase enhanced whole cellulase compositions and methods of use. Generally, the beta-glucosidase enhanced whole cellulase compositions have equal or greater specific performance relative to whole cellulase preparations alone. In some embodiments, the beta-glucosidase enhanced whole cellulase compositions comprise greater than 10% to about 80% (w/w protein) beta-glucosidase. In some embodiments, the beta-glucosidase enhanced whole cellulase compositions comprise a whole cellulase activity and β-glucosidase activity of about 0.60 to 22 pNPG/CMC units.

The present teachings further provide methods of decreasing the amount of a whole cellulase required to hydrolyze a cellulosic material by adding an effective amount β-glucosidase. In some embodiments the method provides decreasing the amount of a whole cellulase required to hydrolyze a cellulosic material by adding an amount of β-glucosidase that is greater than 10% (w/w protein) to amount of the whole cellulase. In some embodiments, the method comprises a whole cellulase activity and β-glucosidase activity wherein the ratio of β-glucosidase activity to cellulase activity is about 0.60 to 22 pNPG/CMC units. The present teachings further provide methods of hydrolyzing a cellulosic material by contacting a cellulosic material with an effective amount of a beta-glucosidase enhanced whole cellulase composition.

These and other features of the present teachings are set forth below.

### 5. BRIEF DESCRIPTION OF THE DRAWINGS

The skilled artisan will understand that the drawings are for illustration purposes only and are not intended to limit the scope of the present teachings in anyway.

FIG. 1 is a graph showing the result of a micro titer plate saccharification assay using a Trichoderma whole cellulase and Trichoderma β-glucosidase 1 on 1% PASC showing the overall % conversion (A) and the relative amounts of cellobiose and glucose produced (B).

FIG. 2 is a graph showing the result of a microtiter plate saccharification assay using a Trichoderma whole cellulase and Trichoderma β-glucosidase 1 on 7% w/w Avicel showing the overall percent conversion (A) and the relative amounts of cellobiose and glucose produced (B).

FIG. 3 is a graph showing the result of a microtiter plate saccharification assay using a Trichoderma whole cellulase and Trichoderma β-glucosidase 1 on 7% w/w PCS showing the overall percent conversion (A) and the relative amounts of cellobiose and glucose produced (B).

FIG. 4 is a graph showing the result of a microtiter plate saccharification assay using a Trichoderma whole cellulase and Trichoderma β-glucosidase 1 on 7% w/w sugarcane bagasse showing the overall percent conversion (A) and the relative amounts of cellobiose and glucose produced (B), and the percent conversion by increasing the amount of beta-glucosidase (C).

FIG. 5 is a graph showing the result of a microtiter plate saccharification assay using Trichoderma whole cellulase Rut C30 and Trichoderma β-glucosidase 1 on 7% w/w PCS showing the overall % conversion (a) and the relative amounts of cellobiose and glucose produced (b).

FIG. 6 is a graph showing the result of a microtiter plate saccharification assay using Trichoderma whole cellulase and purified Trichoderma β-glucosidase 1 on 1% w/w PASC showing the overall % conversion (a) and the relative amounts of cellobiose and glucose produced (b).

FIG. 7 is a graph showing the result of a microtiter plate saccharification assay using Trichoderma whole cellulase and purified Trichoderma β-glucosidase 1 on PCS at 7% w/w showing the overall % conversion (a) and the relative amounts of cellobiose and glucose produced (b).

FIG. 8 is a graph showing the result of a microtiter plate saccharification assay using Trichoderma whole cellulase and purified Trichoderma β-glucosidase 3 on 1% w/w PASC showing the overall % conversion (a) and the relative amounts of cellobiose and glucose produced (b)

FIG. 9 is a graph showing the result of a microtiter plate saccharification assay using Trichoderma whole cellulase and purified Trichoderma β-glucosidase 3 on PCS at 7% w/w showing the overall % conversion (a) and the relative amounts of cellobiose and glucose produced (b).

FIG. 10 is a graph showing the result of a microtiter plate saccharification assay using Trichoderma whole cellulase and purified Trichoderma β-glucosidase 7 on 1% w/w PASC. The overall % conversion is plotted for a given dose of Trichoderma whole cellulase with and without β-glucosidase 7.

### 6. DETAILED DESCRIPTION OF VARIOUS EMBODIMENTS

It is to be understood that both the foregoing general description and the following detailed description are exemplary and explanatory only and are not restrictive of the compositions and methods described herein. Unless defined otherwise herein, all technical and scientific terms used herein have the same meaning as commonly understood by one of ordinary skill in the art to which this invention belongs. In this application, the use of the singular includes the plural unless specifically stated otherwise. The use of “or” means “and/or” unless state otherwise. Likewise, the terms “comprise,” “comprising,” “comprises,” “include,” “including” and “includes” are not intended to be limiting. All patents and publications, including all amino acid and nucleotide sequences disclosed within such patents and publications, referred to herein are expressly incorporated by reference.

The headings provided herein are not limitations of the various aspects or embodiments of the invention which can be had by reference to the specification as a whole. Accordingly, the terms herein are more fully defined by reference to the specification as a whole.

### 6.1. Beta-Glucosidase Enhanced Whole Cellulase Compositions

Beta-glucosidase enhanced whole cellulase compositions are provided, as well as methods of making and using the same. Generally the beta-glucosidase enhanced whole cellulase compositions described herein have about equal or greater specific performance relative to a whole cellulase preparation alone. In some embodiments, the beta-glucosidase enhanced whole cellulase compositions described herein have about equal or greater specific performance in saccharification of cellulosic material relative to a whole cellulase preparation alone.

The beta-glucosidase enhanced whole cellulase compositions can include any polypeptide having beta-glucosidase activity. The term “beta-glucosidase” is defined herein as a beta-D-glucoside glucohydrolase classified as EC 3.2.1.21, and/or those in certain GH families, including, but not limited to, those in GH families 1, 3, 9 or 48, which catalyzes the hydrolysis of cellobiose with the release of beta-D-glucose.

The beta-glucosidase can be obtained from any suitable microorganism, by recombinant means or can be obtained from commercial sources. Suitable, non-limited examples of beta-glucosidase from microorganisms include without limitation bacteria and fungi. Suitable bacteria include Acidothermus, Acetivibrio, Aeromona, Aeromonas, Alicyclobacillus, Anaerocellum, Acinetobacter, Actinobacillus, Alcanivorax, Alkalilimnicola, Alkaliphilus, Anabaena, Arthrobacter, Azoarcus, Azospirillum, Anaeromyxobacter, Butyrivibrio, Bacillus, Bacteroides, Bdellovibrio, Bifidobacterium, Bordetella, Borrelia, Bradyrhizobium, Brucella, Burkholderia, Butyrivibrio, Campylobacter, Caldicellulosiruptor, Caulobacter, Cellvibrio, Chromobacterium, Clavibacter, Colwellia, Corynebacterium, Cyanobacteria, Cytophaga, Eubacterium, Fibrobacter, Flavobacterium, Gloeobacter, Klebsiella, Lactobacillus, Fusobacterium, Hahella, Kineococcus, Lactococcus, Listeria, Maricaulis, Myxobacter, Mesoplasma, Methylococcus, Myxococcus, Microbispora, Oenococcus, Paenibacillus, Photobacterium, Photorhabdus Pectobacterium, Pseudomonas, Ruminococcus, Rhizobium, Rhodobacter, Rhodococcus, Rhodoferax, Rhodopseudomonas, Saccharophagus, Salinispora, Salmonella, Solibacter, Synechocystis, Serratia, Shewanella, Sphingomonas, Staphylococcus, Streptococcus, Streptomyces, Thermotoga, Thermus, Treponema, Thermobifida, Vibrio, Xanthomonas, Acidovorax, Deinococcus geothermalis, Desulfotalea , Enterococcus, Erwinia, and Yersinia.

In some embodiment, beta-glucosidase is obtained from a filamentous fungi. The term “filamentous fungi” means any and all filamentous fungi recognized by those of skill in the art. In general, filamentous fungi are eukaryotic microorganisms and include all filamentous forms of the subdivision Eumycotina and Oomycota. These fungi are characterized by a vegetative mycelium with a cell wall composed of chitin, beta-glucan, and other complex polysaccharides. In some embodiments, the filamentous fungi of the present teachings are morphologically, physiologically, and genetically distinct from yeasts. In some embodiments, the filamentous fungi include, but are not limited to the following genera: Aspergillus, Acremonium, Aureobasidium, Beauveria, Cephalosporium, Ceriporiopsis, Chaetomium paecilomyces, Chrysosporium, Claviceps, Cochiobolus, Cryptococcus, Cyathus, Endothia, Endothia mucor, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, and Trametes pleurotus. In some embodiments, the filamentous fungi include, but are not limited to the following: A. nidulans, A. niger, A. awomari, A. aculeatus, A. kawachi e.g., NRRL 3112, ATCC 22342 (NRRL 3112), ATCC 44733, ATCC 14331 and strain UVK 143f, A. oryzae, e.g., ATCC 11490, Penicillium N. crassa, Trichoderma reesei, e.g., NRRL 15709, ATCC 13631, 56764, 56765, 56466, 56767, and Trichoderma viride, e.g., ATCC 32098 and 32086.

Preferred examples of beta-glucosidase that can be used include beta-glucosidase from Aspergillus aculeatus (Kawaguchi et al., 1996, Gene 173: 287-288), Aspergillus kawachi (Iwashita et al., 1999, Appl. Environ. Microbiol. 65: 5546-5553), Aspergillus oryzae (WO 2002/095014), Cellulomonas biazotea (Wong et al., 1998, Gene 207: 79-86), Penicillium funiculosum (WO 200478919), Saccharomycopsis fibuligera (Machida et al., 1988, Appl. Environ. Microbiol. 54: 3147-3155), Schizosaccharomyces pombe (Wood et al., 2002, Nature 415: 871-880), and Trichoderma reesei beta-glucosidase 1 (U.S. Pat. No. 6,022,725), Trichoderma reesei beta-glucosidase 3 (U.S. Pat. No. 6,982,159), Trichoderma reesei beta-glucosidase 4 (U.S. Pat. No. 7,045,332), Trichoderma reesei beta-glucosidase 5 (U.S. Pat. No. 7,005,289), Trichoderma reesei beta-glucosidase 6 (USPN 20060258554) Trichoderma reesei beta-glucosidase 7 (USPN 20040102619).

In some embodiments, the beta-glucosidase can be produced by expressing a gene encoding beta-glucosidase. For example, beta-glucosidase can be secreted into the extracellular space e.g., by Gram-positive organisms, (such as Bacillus and Actinomycetes), or eukaryotic hosts (e.g., Trichoderma, Aspergillus, Saccharomyces, and Pichia).

It is to be understood, that in some embodiments, that beta-glucosidase can be over-expressed in a recombinant microorganism relative to the native levels. In some embodiments, if a host cell is employed for expression of the beta-glucosidase, the cell may be genetically modified to reduce expression of one or more proteins that are endogenous to the cell. In one embodiment, the cell may contain one or more native genes, particularly genes that encode secreted proteins, that have been deleted or inactivated. For example, one or more protease-encoding genes (e.g. an aspartyl protease-encoding gene; see Berka et al, Gene 1990 86:153-162 and U.S. Pat. No. 6,509,171) or cellulase-encoding genes may be deleted or inactivated. In one embodiment, the Trichoderma sp. host cell may be a T. reesei host cell contain inactivating deletions in the cbh1, cbh2 and egl1, and egl2 genes, as described in WO 05/001036. The nucleic acids encoding beta-glucosidase may be present in the nuclear genome of the Trichoderma sp. host cell or may be present in a plasmid that replicates in the Trichoderma host cell, for example.

The beta-glucosidase can be used as is or the beta-glucosidase may be purified. The term “as is” as used herein refers to an enzyme preparation produced by fermentation that undergoes no or minimal recovery and/or purification. For example, once the beta-glucosidase is secreted by a cell into the cell culture medium, the cell culture medium containing beta-glucosidase can be used. Alternatively, the beta-glucosidase can be recovered from the cell culture medium by any convenient method, e.g., by precipitation, centrifugation, affinity, filtration or any other method known in the art, including Chen, H.; Hayn, M.; Esterbauer, H. “Purification and characterization of two extracellular b-glucosidases from Trichoderma reesei”, Biochimica et Biophysica Acta, 1992, 1121, 54-60. For example, affinity chromatography (Tilbeurgh et al., (1984) FEBS Lett. 16:215); ion-exchange chromatographic methods (Goyal et al., (1991) Biores. Technol. 36:37; Fliess et al., (1983) Eur. J. Appl. Microbiol. Biotechnol. 17:314; Bhikhabhai et al., (1984) J. Appl. Biochem. 6:336; and Ellouz et al., (1987) Chromatography 396:307), including ion-exchange using materials with high resolution power (Medve et al., (1998) J. Chromatography A 808:153; hydrophobic interaction chromatography (Tomaz and Queiroz, (1999) J. Chromatography A 865:123; two-phase partitioning (Brumbauer, et al., (1999) Bioseparation 7:287); ethanol precipitation; reverse phase HPLC; chromatography on silica or on a cation-exchange resin such as DEAE; chromatofocusing; ammonium sulfate precipitation; or gel filtration using, e.g., Sephadex G-75, may be employed.

In some embodiments, the beta-glucosidase can be used without purification from the other components of the cell culture medium. In some embodiments, the cell culture medium can be concentrated, for example, and then used without further purification of the protein from the components of the cell culture medium, or used without any further modification.

Where the beta-glucosidase is obtained from a microorganism, the enzyme can be recovered using recovery methods well known in the art. For example, the enzyme may be recovered from a cell culture medium by conventional procedures including, but not limited to, centrifugation, filtration, extraction, spray-drying, evaporation, or precipitation. In some embodiments, a purified beta-glucosidase can be used. The term “purified beta-glucosidase” as used herein means beta-glucosidase that is free from other components from the organism from which it is obtained. The beta-glucosidase can be purified, with only minor amounts of other proteins being present. The term “purified” as used herein also refers to removal of other components, particularly other enzymes present in the cell of origin of the beta-glucosidase. In some embodiments, beta-glucosidase can be “substantially pure,” that is, free from other components from the microorganism in which it is produced. The beta-glucosidase can be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), or extraction. In some embodiments, the beta-glucosidase is at least 25% pure, preferably at least 50% pure, more preferably at least 75% pure, even more preferably at least 90% pure, most preferably at least 95% pure, and even most preferably at least 99% pure, as determined by SDS-PAGE.

The beta-glucosidase can also be obtained from commercial sources. Examples of commercial beta-glucosidase preparation suitable for use in the present invention include, for example, NOVOZYM™ 188, (a beta-glucosidase from Aspergillus niger), Agrobacterium sp., and Thermatoga maritime available from Megazyme (Megazyme International Ireland Ltd., Bray Business Park, Bray, Co. Wicklow, Ireland.

Beta-glucosidase enhanced whole cellulases generally comprise beta-glucosidase and a whole cellulase preparation. However, it is to be understood that the beta-glucosidase enhanced whole cellulase compositions can be produced by recombinant means. For example, expressing beta-glucosidase in microorganism capable of producing a whole cellulase.

In some embodiments the beta-glucosidase enhanced whole cellulase composition comprises a whole cellulase preparation and beta-glucosidase. Also provided are beta-glucosidase enhanced whole cellulase composition comprising a whole cellulase preparation and beta-glucosidase, wherein the comprising greater than 10% beta-glucosidase.

In some embodiments the beta-glucosidase enhanced whole cellulase composition comprises a whole cellulase preparation and beta-glucosidase, wherein the amount of a whole cellulase preparation required to hydrolyze a cellulosic material to soluble sugars is reduced by the beta-glucosidase.

The beta-glucosidase is generally present in the compositions in an amount relative to the amount of whole cellulase preparation. In some embodiments, the composition comprises a whole cellulase preparation and beta-glucosidase, wherein the beta-glucosidase is present in an amount relative to the amount of whole cellulase preparation on a weight:weight ratio, such as protein:protein ratio.

In some embodiments, the composition comprises a whole cellulase preparation and beta-glucosidase, wherein the amount of beta-glucosidase is in the range of greater than 10% to 90%, relative to total protein, e.g., 11% to 90%, 15% to 85%, 20% to 80%, 25% to 75%, 30% to 70%, 35% to 65%, 40% to 60%, 45% to 55%, and 50% relative to total protein example.

In some embodiments, the compositions comprises a whole cellulase preparation and beta-glucosidase wherein the amount of beta-glucosidase is greater than 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% or greater of the relative to total protein, for example.

As described above, in some embodiments, the compositions generally comprise beta-glucosidase and a whole cellulase preparation. As used herein, the phrase “whole cellulase preparation” refers to both naturally occurring and non-naturally occurring cellulase containing compositionsA “naturally occurring” composition is one produced by a naturally occurring source and which comprises one or more cellobiohydrolase-type, one or more endoglucanase-type, and one or more beta-glucosidase components wherein each of these components is found at the ratio produced by the source. A naturally occurring composition is one that is produced by an organism unmodified with respect to the cellulolytic enzymes such that the ratio of the component enzymes is unaltered from that produced by the native organism. A “non-naturally occurring” composition encompasses those compositions produced by: (1) combining component cellulolytic enzymes either in a naturally occurring ratio or non-naturally occurring, i.e., altered, ratio; or (2) modifying an organism to overexpress or underexpress one or more cellulolytic enzyme; or (3) modifying an organism such that at least one cellulolytic enzyme is deleted. Accordingly, in some embodiments, the whole cellulase preparation can have one or more of the various EGs and/or CBHs, and/or beta-glucosidase deleted. For example, EG1 may be deleted alone or in combination with other EGs and/or CBHs.

In general, the whole cellulase preparation includes enzymes including, but are not limited to: (i) endoglucanases (EG) or 1,4-β-d-glucan-4-glucanohydrolases (EC 3.2.1.4), (ii) exoglucanases, including 1,4-β-d-glucan glucanohydrolases (also known as cellodextrinases) (EC 3.2.1.74) and 1,4-β-d-glucan cellobiohydrolases (exo-cellobiohydrolases, CBH) (EC 3.2.1.91), and (iii) β-glucosidase (BG) or β-glucoside glucohydrolases (EC 3.2.1.21).

In the present disclosure, the whole cellulase preparation can be from any microorganism that is useful for the hydrolysis of a cellulosic material. In some embodiments, the whole cellulase preparation is a filamentous fungi whole cellulase. “Filamentous fungi” include all filamentous forms of the subdivision Eumycota and Oomycota.

In some embodiments, the whole cellulase preparation is a Acremonium, Aspergillus, Emericella, Fusarium, Humicola, Mucor, Myceliophthora, Neurospora, Penicillium, Scytalidium, Thielavia, Tolypocladium, or Trichoderma species, whole cellulase.

In some embodiments, the whole cellulase preparation is an Aspergillus aculeatus, Aspergillus awamori, Aspergillus foetidus, Aspergillus japonicus, Aspergillus nidulans, Aspergillus niger, or Aspergillus oryzae whole cellulase. In another aspect, whole cellulase preparation is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum whole cellulase. In another aspect, the whole cellulase preparation is a Humicola insolens, Humicola lanuginosa, Mucor miehei, Myceliophthora thermophila, Neurospora crassa, Penicillium purpurogenum, Penicillium funiculosum, Scytalidium thermophilum, or Thielavia terrestris whole cellulase. In another aspect, the whole cellulase preparation a Trichoderma harzianum, Trichoderma koningii, Trichoderma longibrachiatum, Trichoderma reesei e.g., RL-P37 (Sheir-Neiss et al., Appl. Microbiol. Biotechnology, 20 (1984) pp. 46-53; Montenecourt B.S., Can., 1-20, 1987), QM9414 (ATCC No. 26921), NRRL 15709, ATCC 13631, 56764, 56466, 56767, or Trichoderma viride e.g., ATCC 32098 and 32086, whole cellulase.

In some embodiments, the whole cellulase preparation is a Trichoderma reesei RutC30 whole cellulase, which is available from the American Type Culture Collection as Trichoderma reesei ATCC 56765.

In some embodiments, the whole cellulase is Penicillium funiculosum, which is available from the American Type Culture Collection as Penicillium funiculosum ATCC Number: 10446. The whole cellulase preparation may also be obtained from commercial sources. Examples of commercial cellulase preparations suitable for use in the present invention include, for example, CELLUCLAST™ (available from Novozymes A/S) and LAMINEX™, IndiAge™ and Primafast™ (available Genencor Division, Danisco US. Inc.)

In the present disclosure, the whole cellulase preparation can be from any microorganism cultivation method known in the art resulting in the expression of enzymes capable of hydrolyzing a cellulosic material. Fermentation can include shake flask cultivation, small- or large-scale fermentation, such as continuous, batch, fed-batch, or solid state fermentations in laboratory or industrial fermenters performed in a suitable medium and under conditions allowing the cellulase to be expressed or isolated.

Generally, the microorganism is cultivated in a cell culture medium suitable for production of enzymes capable of hydrolyzing a cellulosic material. The cultivation takes place in a suitable nutrient medium comprising carbon and nitrogen sources and inorganic salts, using procedures known in the art. Suitable culture media, temperature ranges and other conditions suitable for growth and cellulase production are known in the art. As a non-limiting example, the normal temperature range for the production of cellulases by Trichoderma reesei is 24° C. to 28° C.

Generally, the whole cellulase preparation is used as is produced by fermentation with no or minimal recovery and/or purification. For example, once cellulases are secreted by a cell into the cell culture medium, the cell culture medium containing the cellulases can be used. In some embodiments the whole cellulase preparation comprises the unfractionated contents of fermentation material, including cell culture medium, extracellular enzymes and cells. Alternatively, the whole cellulase preparation can be processed by any convenient method, e.g., by precipitation, centrifugation, affinity, filtration or any other method known in the art. In some embodiments, the whole cellulase preparation can be concentrated, for example, and then used without further purification. In some embodiments the whole cellulase preparation comprises chemical agents that decrease cell viability or kills the cells. In some embodiments, the cells are lysed or permeabilized using methods known in the art.

In some embodiments, the beta-glucosidase enhanced whole cellulase comprises a whole cellulase preparation and beta-glucosidase, wherein the amount of whole cellulase is in the range of less than 90% to 10% relative to total protein, e.g., 89% to 10%, 85% to 15%, 80% to 20%, 75% to 25%, 65% to 30%, 60% to 35%, 65% to 40%, 60% to 45%, 55% to 50% relative to total protein for example.

In some embodiments, the beta-glucosidase enhanced whole cellulase comprises a whole cellulase preparation and beta-glucosidase wherein the concentration of whole cellulase preparation is less than 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, relative to total protein, for example.

In some embodiments, the beta-glucosidase enhanced whole cellulase composition comprises a whole cellulase preparation and beta-glucosidase, wherein the amount of beta-glucosidase is in the range of 10% to 90% of the total protein and the whole cellulase comprises less than 90% to 10% of total protein, for example, the beta-glucosidase comprises 11% and the whole cellulase comprises 89% of total protein, beta-glucosidase comprises 12% and the whole cellulase comprises 88% of total protein, beta-glucosidase comprises 13% and the whole cellulase comprises 87% of total protein, beta-glucosidase comprises 14% and the whole cellulase comprises 86% of total protein, beta-glucosidase comprises 15% and the whole cellulase comprises 85% of total protein, beta-glucosidase comprises 16% and the whole cellulase comprises 84% of total protein, beta-glucosidase comprises 17% and the whole cellulase comprises 83% of total protein, beta-glucosidase comprises 18% and the whole cellulase comprises 82% of total protein, beta-glucosidase comprises 19% and the whole cellulase comprises 81% of total protein, beta-glucosidase comprises 20% and the whole cellulase comprises 80% of total protein, beta-glucosidase comprises 21% and the whole cellulase comprises 79% of total protein, beta-glucosidase comprises 22% and the whole cellulase comprises 78% of total protein, beta-glucosidase comprises 23% and the whole cellulase comprises 77% of total protein, beta-glucosidase comprises 24% and the whole cellulase comprises 76% of total protein, beta-glucosidase comprises 25% and the whole cellulase comprises 75% of total protein, beta-glucosidase comprises 26% and the whole cellulase comprises 74% of total protein, beta-glucosidase comprises 27% and the whole cellulase comprises 73% of total protein, beta-glucosidase comprises 28% and the whole cellulase comprises 72% of total protein, beta-glucosidase comprises 29% and the whole cellulase comprises 71% of total protein, beta-glucosidase comprises 30% and the whole cellulase comprises 70% of total protein, beta-glucosidase comprises 31% and the whole cellulase comprises 69% of total protein, beta-glucosidase comprises 32% and the whole cellulase comprises 68% of total protein, beta-glucosidase comprises 33% and the whole cellulase comprises 67% of total protein, beta-glucosidase comprises 34% and the whole cellulase comprises 66% of total protein, beta-glucosidase comprises 35% and the whole cellulase comprises 65% of total protein, beta-glucosidase comprises 36% and the whole cellulase comprises 64% of total protein, beta-glucosidase comprises 37% and the whole cellulase comprises 63% of total protein, beta-glucosidase comprises 38% and the whole cellulase comprises 62% of total protein, beta-glucosidase comprises 39% and the whole cellulase comprises 61% of total protein, beta-glucosidase comprises 40% and the whole cellulase comprises 60% of total protein, beta-glucosidase comprises 41% and the whole cellulase comprises 59% of total protein, beta-glucosidase comprises 42% and the whole cellulase comprises 58% of total protein, beta-glucosidase comprises 43% and the whole cellulase comprises 57% of total protein, beta-glucosidase comprises 44% and the whole cellulase comprises 56% of total protein, beta-glucosidase comprises 45% and the whole cellulase comprises 55% of total protein, beta-glucosidase comprises 46% and the whole cellulase comprises 54% of total protein, beta-glucosidase comprises 47% and the whole cellulase comprises 53% of total protein, beta-glucosidase comprises 48% and the whole cellulase comprises 52% of total protein, beta-glucosidase comprises 49% and the whole cellulase comprises 51% of total protein, beta-glucosidase comprises 50% and the whole cellulase comprises 50% of total protein, beta-glucosidase comprises 51% and the whole cellulase comprises 49% of total protein, beta-glucosidase comprises 52% and the whole cellulase comprises 48% of total protein, beta-glucosidase comprises 53% and the whole cellulase comprises 47% of total protein, beta-glucosidase comprises 54% and the whole cellulase comprises 46% of total protein, beta-glucosidase comprises 55% and the whole cellulase comprises 45% of total protein, beta-glucosidase comprises 56% and the whole cellulase comprises 44% of total protein, beta-glucosidase comprises 57% and the whole cellulase comprises 43% of total protein, beta-glucosidase comprises 58% and the whole cellulase comprises 42% of total protein, beta-glucosidase comprises 59% and the whole cellulase comprises 41% of total protein, beta-glucosidase comprises 60% and the whole cellulase comprises 40% of total protein, beta-glucosidase comprises 61% and the whole cellulase comprises 39% of total protein, beta-glucosidase comprises 62% and the whole cellulase comprises 38% of total protein, beta-glucosidase comprises 63% and the whole cellulase comprises 37% of total protein, beta-glucosidase comprises 64% and the whole cellulase comprises 36% of total protein, beta-glucosidase comprises 65% and the whole cellulase comprises 35% of total protein, beta-glucosidase comprises 66% and the whole cellulase comprises 34% of total protein, beta-glucosidase comprises 67% and the whole cellulase comprises 33% of total protein, beta-glucosidase comprises 68% and the whole cellulase comprises 32% of total protein, beta-glucosidase comprises 69% and the whole cellulase comprises 31% of total protein, beta-glucosidase comprises 70% and the whole cellulase comprises 20% of total protein, beta-glucosidase comprises 71% and the whole cellulase comprises 29% of total protein, beta-glucosidase comprises 72% and the whole cellulase comprises 28% of total protein, beta-glucosidase comprises 73% and the whole cellulase comprises 27% of total protein, beta-glucosidase comprises 74% and the whole cellulase comprises 26% of total protein, beta-glucosidase comprises 75% and the whole cellulase comprises 25% of total protein, beta-glucosidase comprises 76% and the whole cellulase comprises 24% of total protein, beta-glucosidase comprises 77% and the whole cellulase comprises 23% of total protein, beta-glucosidase comprises 78% and the whole cellulase comprises 22% of total protein, beta-glucosidase comprises 79% and the whole cellulase comprises 21% of total protein, beta-glucosidase comprises 80% and the whole cellulase comprises 20% of total protein, beta-glucosidase comprises 81% and the whole cellulase comprises 19% of total protein, beta-glucosidase comprises 82% and the whole cellulase comprises 18% of total protein, beta-glucosidase comprises 83% and the whole cellulase comprises 17% of total protein, beta-glucosidase comprises 84% and the whole cellulase comprises 16% of total protein, beta-glucosidase comprises 85% and the whole cellulase comprises 15% of total protein, beta-glucosidase comprises 86% and the whole cellulase comprises 14% of total protein, beta-glucosidase comprises 87% and the whole cellulase comprises 13% of total protein, beta-glucosidase comprises 88% and the whole cellulase comprises 12% of total protein, beta-glucosidase comprises 89% and the whole cellulase comprises 11% of total protein, beta-glucosidase comprises 90% and the whole cellulase comprises 10% of total protein.

In some embodiments, the beta-glucosidase enhanced whole cellulase comprises a whole cellulase preparation and beta-glucosidase, wherein the amount of beta-glucosidase is approximately equal to the amount of whole cellulase preparation on a weight:weight ratio. In some embodiments, the beta-glucosidase enhanced whole cellulase comprises a whole cellulase preparation and beta-glucosidase, wherein the amount of beta-glucosidase is about 50% to the amount of whole cellulase preparation on a weight:weight ratio.

As described above, the beta-glucosidase is generally present in the compositions in an amount relative to the amount of whole cellulase preparation. In some embodiments, the composition comprises a whole cellulase preparation and beta-glucosidase, wherein the beta-glucosidase is present in an amount relative to the amount of whole cellulase preparation based on enzyme activity. In some embodiments, the compositions according to the invention can be characterized by a relation between the activity of the beta-glucosidase and the activity of the whole cellulase preparation. In some embodiments, the composition comprises a whole cellulase preparation and beta-glucosidase, wherein the beta-glucosidase activity and activity of the whole cellulase preparation are provided as a ratio of enzymatic activity.

The above-mentioned enzyme activity ratios relate to the respective standard assay conditions for the beta-glucosidase and whole cellulase preparations. The activity of the beta-glucosidase and the activity of the whole cellulase preparation can be determined using methods known in the art. In this context, the following conditions can be used. Beta-glucosidase activity can determined by any means know in the art, such as the assay described by Chen, H.; Hayn, M.; Esterbauer, H. “Purification and characterization of two extracellular b-glucosidases from Trichoderma reesei”, Biochimica et Biophysica Acta, 1992, 1121, 54-60. One pNPG denotes 1 μmol of Nitrophenol liberated from para-nitrophenyl-B-D-glucopyranoside in 10 minutes at 50° C. (122° F.) and pH 4.8. Cellulase activity of the whole cellulase preparation may be determined using carboxymethyl cellulose (CMC) as a substrate. Determination of whole cellulase activity, measured in terms of CMC activity. This method measures the production of reducing ends created by the enzyme mixture acting on CMC wherein 1 unit is the amount of enzyme that liberates 1 μmol of product/minute (Ghose, T. K., Measurement of Cellulse Activities, Pure & Appl. Chem. 59, pp. 257-268, 1987).

In general, the beta-glucosidase enhanced whole cellulase comprise an enzyme activity ratio in a range from about 0.5 to 25 pNPG/CMC units. In some embodiments, enzyme activity ratio is from about 1 to 20 pNPG/CMC units, or from about 1.5 to 15 pNPG/CMC units, or from about 2 to 10 pNPG/CMC units, or from about 2.5 to 8 pNPG/CMC units, from about 3 to 7 pNPG/CMC units, or from about 3.5 to 6.5 pNPG/CMC units, or from about 4 to 6 pNPG/CMC unit, or from about 4.5 to 5.5 pNPG/CMC units, or from about 5 to 6 pNPG/CMC. Especially suitable are, for example, ratios of about 5.5 pNPG/CMC units.

### 6.2. Methods

In addition to the above-described beta-glucosidase enhanced whole cellulase compositions, methods use of the compositions described herein are also provided. In a general aspect, the present teachings concerns hydrolyzing a cellulosic materials. These methods generally include contacting a cellulosic material with a beta-glucosidase enhanced whole cellulase and maintaining the cellulosic material and beta-glucosidase enhanced whole cellulase together under conditions sufficient to effect the hydrolysis of the cellulosic material and thereby produce a product. In some embodiments, methods of converting cellulose to glucose are provided.

Generally the beta-glucosidase enhanced whole cellulase compositions have about equal or greater specific performance than a whole cellulase preparation alone. The methods described herein are generally more cost effective than equivalent methods using whole cellulase alone. In particular embodiments, in comparison to otherwise equivalent methods using a whole cellulase alone, the beta-glucosidase enhanced whole cellulase and methods described herein require less whole cellulase protein to hydrolyze a cellulosic material. Using a saccarification assay, for example, the subject methods decreased the amount of whole cellulase required to hydrolyze a cellulosic material by about one-half than an equivalent method with whole cellulase alone. In particular embodiments, in comparison to otherwise equivalent methods using a whole cellulase alone, the beta-glucosidase enhanced whole cellulase and methods described herein require less whole cellulase activity to hydrolyze a cellulosic material. Using a saccarification assay, for example, the subject methods decreased the amount of whole cellulase activity required to hydrolyze a cellulosic material by about one-half than an equivalent method with whole cellulase alone.

Provided herein are methods of decreasing the amount of a whole cellulase preparation required to hydrolyze a cellulosic material by adding an effective amount beta-glucosidase and the amount of said whole cellulase preparation required to hydrolyze said cellulosic material is decreased. In some embodiments, the beta-glucosidase enhanced whole cellulase has about equal or greater specific performance specific performance relative to said whole cellulase preparation alone. Generally the amount of beta-glucosidase is greater than 10% of the amount of the whole cellulase on a weight:weight ratio. In some embodiments, the ratio of beta-glucosidase activity to whole cellulase activity is greater than 0.61 pNPG/CMC units.

Means of detecting a decrease in the whole cellulase required to hydrolyze a cellulosic material are know in the art, for example, a saccarification assay. In some embodiments, the method of decreasing the amount of a whole cellulase preparation required to hydrolyze a cellulosic material by adding an effective amount beta-glucosidase, is provided, wherein, beta-glucosidase decreases the amount of whole cellulase required to hydrolyze over 30% of the cellulosic material in about 48 hrs at 50° C.

Also provided are methods of hydrolyzing a cellulosic material comprising contacting a cellulosic material with an effective amount of beta-glucosidase and a whole cellulase composition, wherein the amount of beta-glucosidase decreases the amount of the whole cellulase composition required to hydrolyze a cellulosic material in some embodiments, the amount of beta-glucosidase is greater than 10% of the amount of the whole cellulase on a weight:weight ratio. In some embodiments, wherein the amount of beta-glucosidase is less than 80% of the amount of whole cellulase on a weight:weight ratio. In some embodiments, the ratio of beta-glucosidase activity to whole cellulase activity is greater than 0.61 pNPG/CMC units.

The beta-glucosidase is generally in an amount relative to the amount of whole cellulase preparation. In some embodiments, the beta-glucosidase is present in an amount relative to the amount of whole cellulase preparation on weight:weight ratio, such as protein:protein ratio. In some embodiments, amount of beta-glucosidase is in the range of greater than 10% to 90%, relative to total protein, e.g., 11% to 90%, 15% to 85%, 20% to 80%, 25% to 75%, 30% to 70%, 35% to 65%, 40% to 60%, 45% to 55%, and 50% relative to total protein example.

In some embodiments, the amount of beta-glucosidase is greater than 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% or greater of the relative to total protein, for example.

In some embodiments, the amount of beta-glucosidase in the method is provided in relation between the activity of the beta-glucosidase and the activity of the whole cellulase preparation. In some embodiments, amount of beta-glucosidase activity in the method is provided as enzyme activity relative to the enzyme activity of the whole cellulase preparation. In general, the enzyme activity ratios of the beta-glucosidase to the whole cellulase preparation in are in a range from about 0.5 to 25 pNPG/CMC units. In some embodiments, enzyme activity ratio is from about 1 to 20 pNPG/CMC units, or from about 1.5 to 15 pNPG/CMC units, or from about 2 to 10 pNPG/CMC units, or from about 2.5 to 8 pNPG/CMC units, from about 3 to 7 pNPG/CMC units, or from about 3.5 to 6.5 pNPG/CMC units, or from about 4 to 6 pNPG/CMC unit, or from about 4.5 to 5.5 pNPG/CMC units, or from about 5 to 6 pNPG/CMC. Especially suitable are, for example, ratios of about 5.5 pNPG/CMC units.

The compositions described herein can be are added in amounts effective from about 0.001 to 10.0% wt. of solids, more preferably from about 0.025% to 4.0% wt. of solids, and most preferably from about 0.005% to 5.0% wt. of solids.

In the methods of the present disclosure, cellulosic material can be any cellulose containing material. The cellulosic material can include, but is not limited to, cellulose, and hemicellulose. In some embodiments, the cellulosic materials include, but are not limited to, biomass, herbaceous material, agricultural residues, forestry residues, municipal solid waste, waste paper, and pulp and paper residues.

In some embodiments, the cellulosic material includes wood, wood pulp, papermaking sludge, paper pulp waste streams, particle board, corn stover, corn fiber, rice, paper and pulp processing waste, woody or herbaceous plants, fruit pulp, vegetable pulp, pumice, distillers grain, grasses, rice hulls, sugar cane bagasse, cotton, jute, hemp, flax, bamboo, sisal, abaca, straw, corn cobs, distillers grains, leaves, wheat straw, coconut hair, algae, switchgrass, and mixtures thereof.

The cellulosic material can be used as is or may be subjected to pretreatment using conventional methods known in the art. Such pretreatments includes chemical, physical, and biological pretreatment. For example, physical pretreatment techniques can include without limitation various types of milling, crushing, steaming/steam explosion, irradiation and hydrothermolysis. Chemical pretreatment techniques can include without limitation dilute acid, alkaline, organic solvent, ammonia, sulfur dioxide, carbon dioxide, and pH-controlled hydrothermolysis. Biological pretreatment techniques can include without limitation applying lignin-solubilizing microorganisms.

The methods of the present disclosure can be used in the production of monosaccharides, disaccharides, and polysaccharides as chemical or fermentation feedstocks for microorganism for the production of organic products, chemicals and fuels, plastics, and other products or intermediates. In particular, the value of processing residues (dried distillers grain, spent grains from brewing, sugarcane bagasse, etc.) can be increased by partial or complete solubilization of cellulose or hemicellulose. In addition to ethanol, some chemicals that can be produced from cellulose and hemicellulose include, acetone, acetate, glycine, lysine, organic acids (e.g., lactic acid), 1,3-propanediol, butanediol, glycerol, ethylene glycol, furfural, polyhydroxyalkanoates, cis, cis-muconic acid, animal feed and xylose.

Aspects of the present teachings may be further understood in light of the following examples, which should not be construed as limiting the scope of the present teachings. It will be apparent to those skilled in the art that many modifications, both to materials and methods, may be practiced without departing from the present teachings.

### 7. EXAMPLES

### Example 7.1

### Saccharification Assays Materials and Methods

Whole cellulases and beta-glucosidases used for the assays are as follows: Trichoderma reesei whole cellulase available as LAMINEX BG from Genencor, USA; Trichoderma reesei RUT-C30 whole cellulase (ATCC No. 56765); Trichoderma reesei BGL1 (CEL3A) (See U.S. Pat. No. 6,022,725); Trichoderma reesei BGL3 (CEL3B) (See U.S. Pat. No. No. 6,982,159), and Trichoderma reesei BGL7 (CEL3E) (See USPN 20040102619). All enzymes were diluted to desired concentrations in 50 mM Sodium Acetate, pH 5.

With the exception of Avicel, all substrates were brought to the desired percentage solids prior to use in the assays. PCS and sugarcane bagasse were blended to allow for accurate pipetting into the microtiter plates. Substrate materials used: Pretreated corn stover PCS and sugar cane bagasse were dilute-sulfuric acid pre-treated by U.S. Department of Energy National Renewable Energy Laboratory (NREL), washed and adjusted to pH 5). The acid pre-treated cornstover (PCS) was 56% Cellulose, 4% Hemicellulose, 29% Lignin. The acid pre-treated bagasse (APB) was 53% Cellulose, 3% Hemicellulose, 31% Lignin. Avicel (pure, crystalline cellulose) was added to plate and then diluted appropriately to 7% (7mgs/ml) with 50 mM Sodium Acetate at pH 5. PASC (phosphoric acid swollen cellulose; pure, amorphous cellulose, diluted in 50 mM Sodium Acetate, to 0.5% PASC at pH 5.

Enzymes were dosed based on total protein and total protein was measured using either BCA Protein Assay Kit, Pierce Cat. No. 23225 or biuret method. Total enzyme loading was 20 mg protein per gram of cellulose. Several ratios of whole cellulase preparations to beta-glucosidase were then used, for example 50:50 ratio would be 10 mg/g whole cellulase preparation and 10 mg/g beta-glucosidase.

One hundred fifty microliters of substrate per well was loaded into a flat-bottom 96-well microtiter plate (MTP) using a repeater pipette. Twenty microliters of appropriately diluted enzyme solution was added on top. In the case of PASC, the enzyme was added to the plate first. The plates were covered with aluminum plate sealers and placed in incubators at either 37 or 50° C., with shaking, for the times specified in Table 1. The reaction was terminated by adding 100 μl 100 mM Glycine pH 10 to each well. With thorough mixing, the contents thereof were filtered through a Millipore 96-well filter plate (0.45 μm, PES). The filtrate was diluted into a plate containing 100 μl 10 mM Glycine pH 10 and the amount of soluble sugars produced measured by HPLC. The Agilent 1100 series HPLCs were all equipped with a de-ashing/guard column (Biorad #125-0118) and an Aminex lead based carbohydrate column (Aminex HPX-87P). The mobile phase was water with a 0.6 ml/min flow rate.

For shake flask experiments, dilute acid pretreated corn stover was loaded into 500 ml shake flasks such that the starting hydrolysis would contain 7% cellulose. Four hundred microliters of tetracycline and 300 microliters of cyclohexamide were dosed to prohibit microbial growth. The final working hydrolyzate volume was brought up to 100 ml with buffer (0.1 M Sodium Citrate, pH 4.8). Lastly, the enzyme was added at a constant total protein loading of 20 mg protein/g cellulose before capping each flask tightly and placing into the shaker/incubator. Each enzyme loading was run at 37 and 50° C. and in duplicate for 72 hours at 200 rpm. The soluble sugars produced were measured by HPLC as described above.

### Example 7.2

### Whole Cellulase and Beta-Glucosidase 1 Saccharification Assay on PASC Substrate

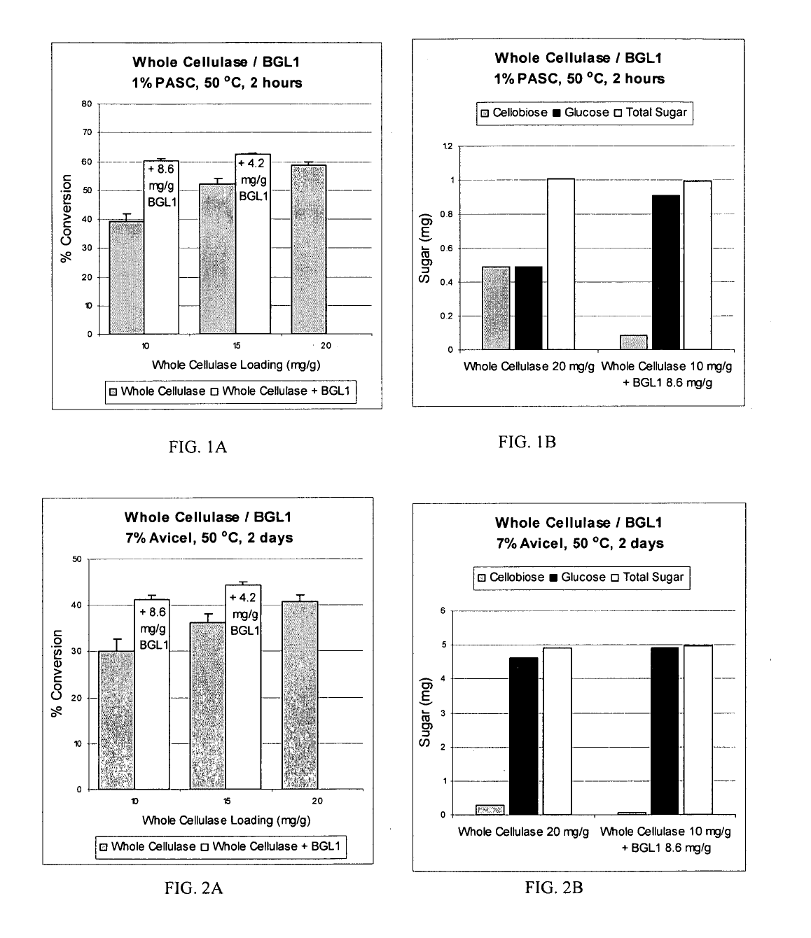

A microtiter plate saccharification assay was carried out using a Trichoderma reesei whole cellulase preparation with and without BGL1 on 1% PASC. FIG. 1 shows a microtiter plate saccharification assay using Trichoderma reesei whole cellulase LAMINEX BG and BGL1 on 1% PASC. FIG. 1(a) shows the overall % conversion plotted for a given dose of whole cellulase with and without BGL1. FIG. 1(b) shows relative amounts of cellobiose and glucose produced by whole cellulase alone and whole cellulase and BGL1 at the same total protein loading.

FIG. 1(a) shows that a the addition of 10 mg/g BGL1 to 10 mg/g whole cellulase converted as much, or more, cellulose to soluble sugars as 20 mg/g whole cellulase. In other words, about one-half of the whole cellulase could be replaced with beta-glucosidase, resulting in an enzyme mixture with equal or better specific performance than whole cellulase alone. Moreover, the product of the whole cellulase beta-glucosidase mixture had a higher proportion of glucose to cellobiose than did the whole cellulase alone when loaded at equal protein. Replacing about one-half the whole cellulase preparation with BGL1 did not affect the overall saccarification rate.

Using methods known in the art a T. reesei whole cellulase producing strain was transformed by electroporation with a polynucleotide encoding T. reesei BGL1 under the CBH2 promoter and with acetamidase (amdS) selection. The stable transformants were grown for one week and evaluated by SDS-PAGE for the level of BGL1 expression. Those transformants which showed high BGL1 expression (about 50% of the total protein) relative to total cellulase protein were tested for activity on phosphoric acid swollen cellulose. The results showed that several of transformants expressing BGL1 had equal or higher specific performance than the T. reesei whole whole cellulase that was not transformed to overexpress BGL1.

### Example 7.3

### Whole Cellulase and Beta-glucosidase 1 Saccharification Assay on Avicel, Pre-Treated Cornstover (PCS) and Sugarcane Bagasse

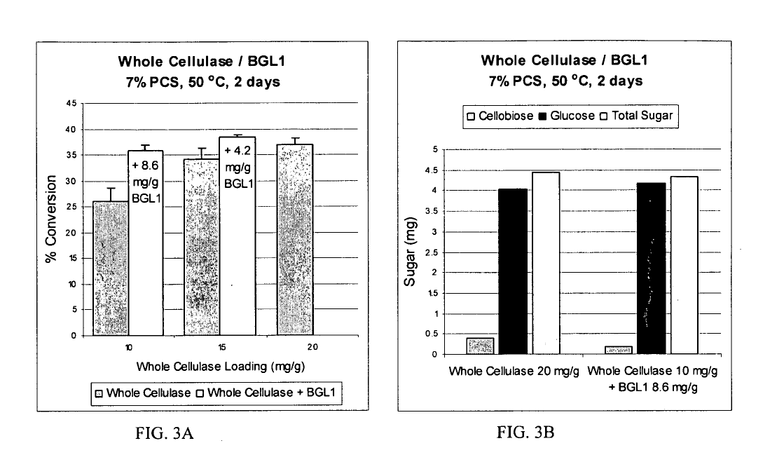

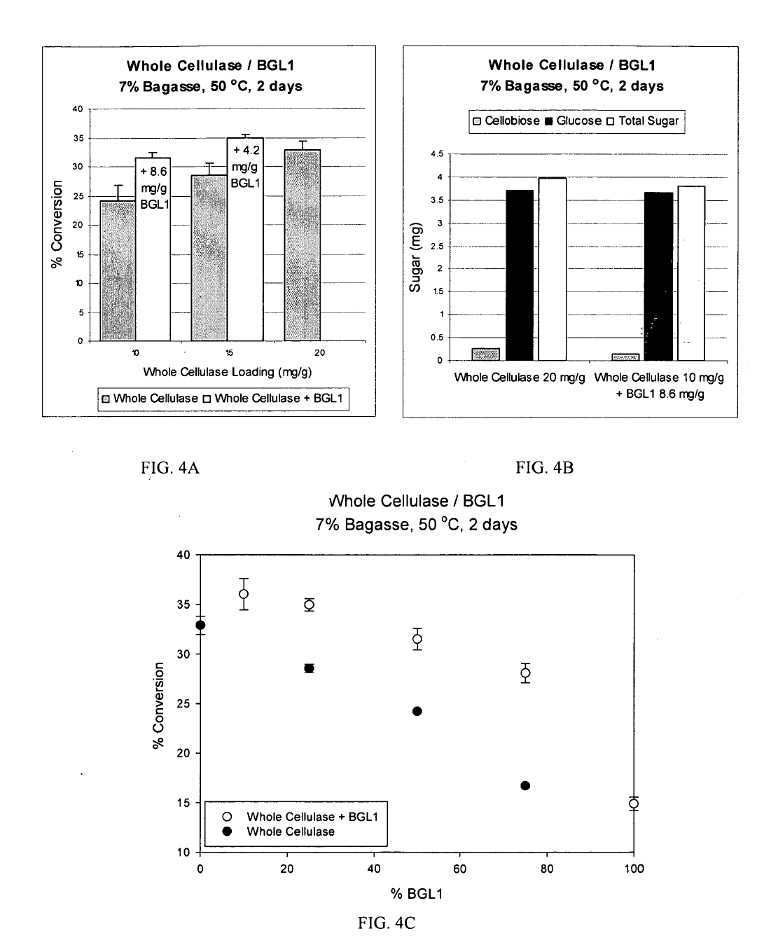

The effect found with the addition BGL1 described in FIG. 1 was not unique to the PASC substrate, which is pure, amorphous cellulose. A similar effect was also observed with other cellulosic materials: crystalline cellulose (Avicel), dilute acid pre-treated cornstover (PCS) and dilute acid pre-treated sugarcane bagasse. FIG. 2 shows the results of experiments, as described above on PASC, performed on Avicel at 7% cellulose solids. FIG. 2 shows microtiter plate saccharification assay using Trichoderma reesei whole cellulase LAMINEX BG and BGL1 on 7% Avicel. FIG. 2(a) shows the overall % conversion plotted for a given dose of whole cellulase with and without BGL1. FIG. 2(b) show he relative amounts of cellobiose and glucose produced by whole cellulase alone and whole cellulase and BGL1 at the same total protein loading. Like with PASC, replacing about half the whole cellulase preparation with BGL1 does not change the overall % conversion. The additional beta-glucosidase increased the ratio of cellobiose to glucose, but the effect is not as pronounced as with PASC as the whole cellulase alone yields a much higher glucose to cellobiose ratio on Avicel. The PCS and bagasse results are similar to those seen with Avicel, in both overall conversion and in the ratio of glucose to cellobiose (FIGS. 3 and 4). FIG. 3 shows microtiter plate saccharification assay using Trichoderma reesei whole cellulase LAMINEX BG and BGL1 on PCS at 7% cellulose: (a) the overall % conversion is plotted for a given dose of whole cellulase with and without BGL1; (b) the relative amounts of cellobiose and glucose produced by whole cellulase alone and whole cellulase and BGL1 at the same total protein loading. Various ratios of whole cellulase to BGL1 on 7% PCS were also tested in shake flasks. The shake flask data correlated well with what was observed in the microtiter plates (data not shown). FIG. 4 is a graph showing the result of a microtiter plate saccharification assay using a Trichoderma whole cellulase and Trichoderma β-glucosidase 1 on 7% sugarcane bagasse showing the overall percent conversion (A) and the relative amounts of cellobiose and glucose produced (B) and the percent conversion by increasing the amount of beta-glucosidase (C).

### Example 7.4.

### Filamentous Fungal Whole Cellulases and Beta-Glucosidase Saccharification Assay on Pre-Treated Corn Stover

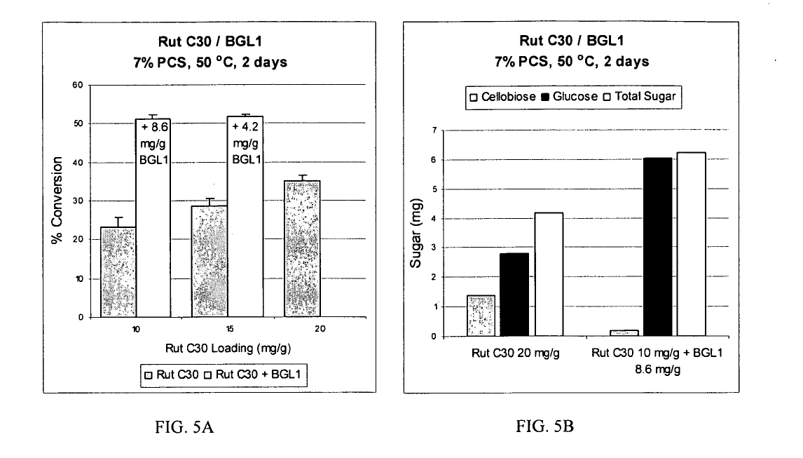

To see if the effect of adding beta-glucosidase was unique to the strain of T. reesei whole cellulase, the experiment with PCS at 7% cellulose solids was repeated with Rut C30 (another T. reesei whole cellulase). FIG. 5 shows a microtiter plate saccharification assay using Rut C30 whole cellulase and BGL1 on PCS at 7% cellulose: (a) the overall % conversion is plotted for a given dose of Rut C30 whole cellulase with and without BGL1; (b) the relative amounts of cellobiose and glucose produced by Rut C30 whole cellulase alone and Rut C30 whole cellulase and BGL1 at the same total protein loading.

At equal protein, Rut C30 whole cellulase does not hydrolyze as much cellulose as either Laminex BG (FIG. 3). When about one-fourth or one-half of the Rut C30 whole cellulase proteins are replaced with BGL1, the % conversion is greater than the same amount of Rut C30 whole cellulase alone (FIG. 5). In this instance, the addition of beta-glucosidase can be used to reduce the overall dose of enzyme required to reach a particular conversion rate.

### Example 7.5

### Whole Cellulase and Purified Beta-Glucosidase 1 Saccharification Assay on PACS and Pre-Treated Cornstover (PCS)

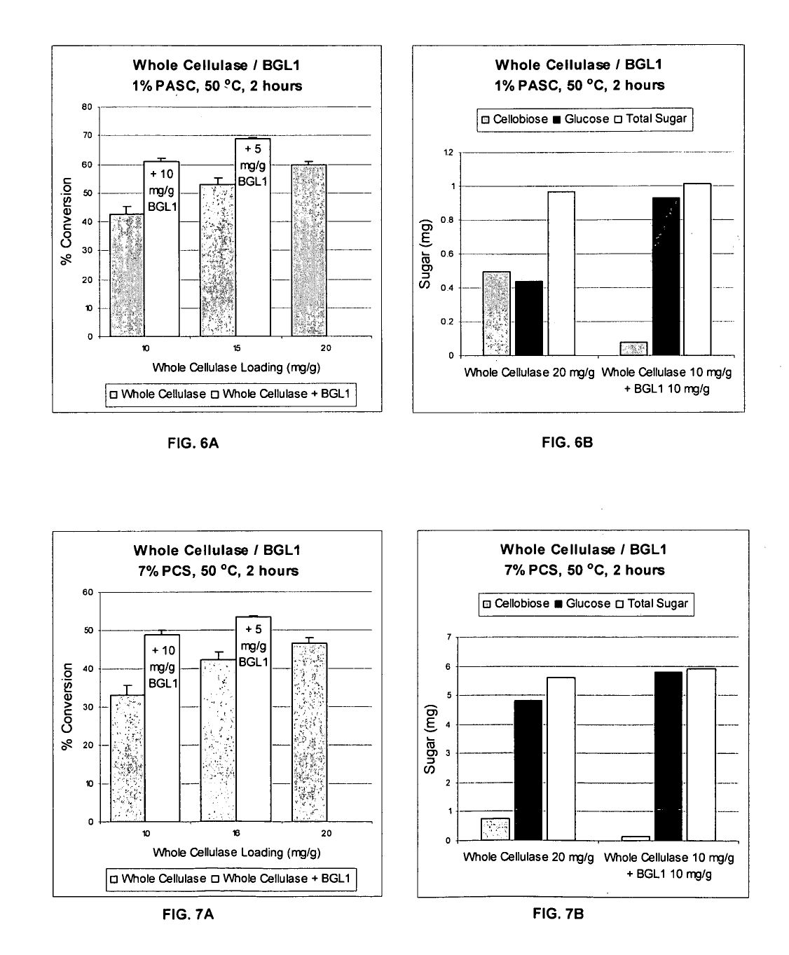

FIG. 6 shows a microtiter plate saccharification assay using Trichoderma reesei whole cellulase LAMINEX BG and purified BGL1 on 1% PASC: (a) the overall % conversion is plotted for a given dose of Trichoderma reesei whole cellulase and BGL1 with and without BGL1; and (b) the relative amounts of cellobiose and glucose produced by Trichoderma reesei whole cellulase and BGL1 at the same total protein loading. FIG. 7 shows a microtiter plate saccharification assay using Trichoderma reesei whole cellulase LAMINEX BG and purified BGL1 on PCS at 7% cellulose: (a) the overall % conversion is plotted for a given dose of Trichoderma reesei whole cellulase with and without BGL1 and (b) the relative amounts of cellobiose and glucose produced by Trichoderma reesei whole cellulase alone and Trichoderma reesei whole cellulase and BGL1 at the same total protein loading. The % conversion from about a 50:50 mixture of BGL1 and whole cellulase, is now higher than the same amount of whole cellulase alone when using either 1% PASC or 7% PCS as the substrate (FIGS. 6 and 7). A mixture of 15 mg/g Trichoderma reesei whole cellulase and 5 mg/g BGL1 also yields higher conversion than 20 mg/g of Trichoderma reesei whole cellulase on both PCS and PASC. As with the unpurified BGL1, the glucose to cellobiose ratio increases with addition of purified BGL1, with a more dramatic difference seen on PASC.

### Example 7.6

### Whole Cellulase and Beta-Glucosidase 3 or Beta-Glucosidase 7 Saccharification Assay on Avicel, Pre-Treated Cornstover (PCS) and Sugarcane Bagasse

The benefit of adding beta-glucosidase to whole cellulase described above is not limited to BGL1. Two other T. reesei beta-glucosidases, BGL3 and BGLI, were also tested with whole cellulase in the microtiter plate saccharification assay on PASC and PCS.

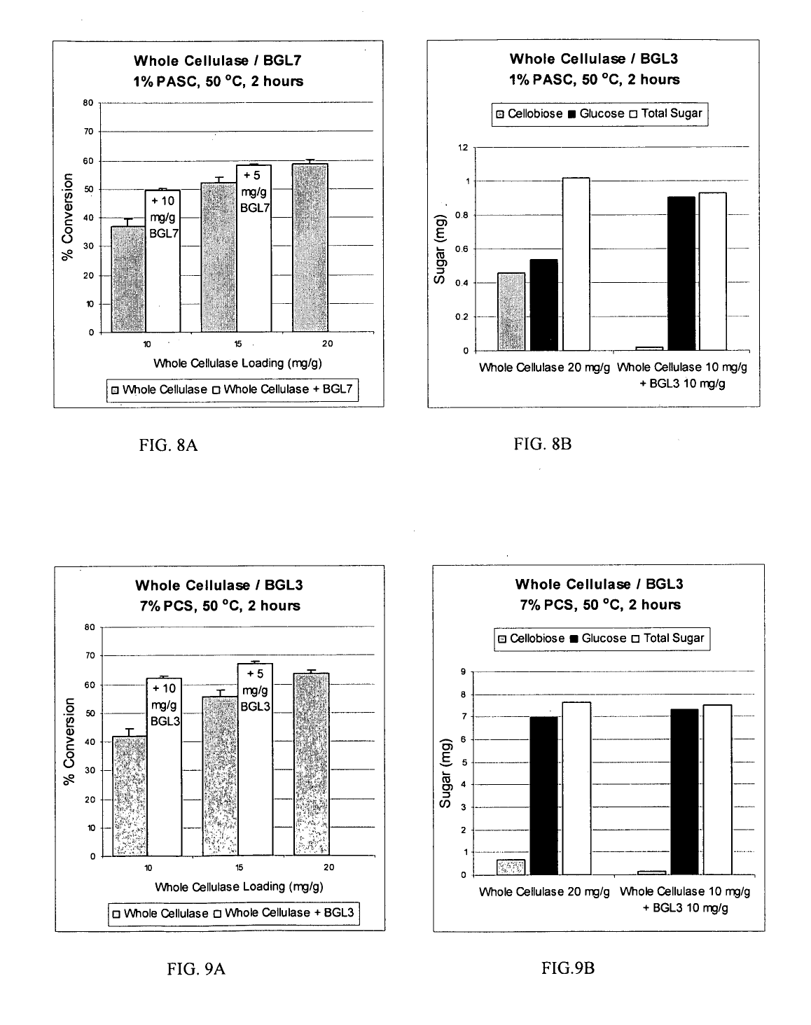

FIG. 8 shows the microtiter plate saccharification assay using Trichoderma reesei whole cellulase Laminex BG and purified BGL3 on 1% PASC. (a) The overall % conversion is plotted for a given dose of Trichoderma reesei whole cellulase with and without BGL3. (b) The relative amounts of cellobiose and glucose produced by Trichoderma reesei whole cellulase alone and Trichoderma reesei whole cellulase and BGL3 at the same total protein loading. The addition of equal parts purified BGL3 to whole cellulase has a similar though slightly less pronounced effect on PASC than that seen with BGL1 (FIG. 6a). While an improvement over 10 mg/g whole cellulase alone, the mixture of 10 mg/g Trichoderma reesei whole cellulase and 10 mg/g BGL3 does not yield equal conversion to 20 mg/g Trichoderma reesei whole cellulase, as seen in the case of BGL1 (FIG. 6a). But the blend of 15 mg/g Trichoderma reesei whole cellulase with 5 mg/g BGL3 does yield a performance benefit over the same total protein loading of Trichoderma reesei whole cellulase alone, though again it not as pronounced a benefit as that seen with BGL1. The replacement of about half the Trichoderma reesei whole cellulase total protein with BGL3 also increases the ratio of glucose to cellobiose; though the total sugar is slightly lower (FIG. 8b). The effect is similar on PCS at 7% cellulose. Replacing about half the total protein with BGL3 results in similar, but not higher % conversion of cellulose to soluble sugars (FIG. 9a); a result similar but less pronounced than that seen with BGL1 (FIG. 7a). The ratio of glucose to cellobiose is also reduced, but the reduction in cellobiose concentration is less. This is not surprising because the total cellobiose produced is lower on PCS than on PASC.

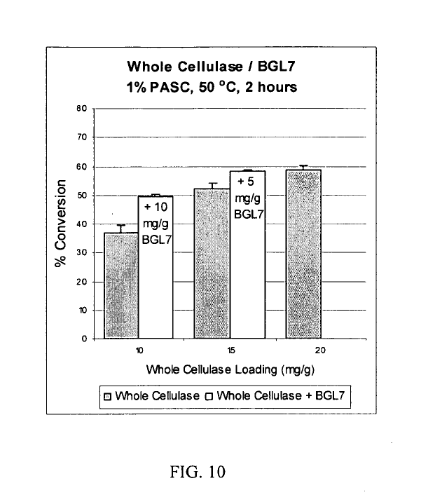

Another T. reesei beta-glucosidase, BGL7, was also tested on 1% PASC in the same manner as BGL1 and BGL3. FIG. 11 shows a microtiter plate saccharification assay using Trichoderma reesei whole cellulase Laminex BG and purified BGL7 on 1% PASC. The overall % conversion is plotted for a given dose of Trichoderma reesei whole cellulase with and without BGL7. Though there is some improvement from adding large amounts of BGL7 (FIG. 10), it is not as pronounced as that seen with BGL1 and BGL7 (FIGS. 6a, 7a). On an equal protein basis, there is no mixture of BGL7 and Trichoderma reesei whole cellulase which yields higher specific performance than the whole cellulase alone.

### Example7.7

### Whole Cellulase Activity and Beta-Glucosidase Activity

Table 1 shows the ratio of activity units for Trichoderma whole cellulase (WC) and beta-glucosidase 1. Enzyme loading in the microtiter plate saccharification assay was converted from mg total protein to units of activity by multiplying by the activity Units/mg protein. Trichoderma reesei whole cellulase 14 CMC U/mg (See Berlin, A.; Maximenko, V.; Gilkes, N.; Saddler, J. “Optimization of enzyme complexes for lignocellulose hydrolysis” Biotechnol. Bioeng. 2007, 97(2), 287-296) while activity of the BGL1 was measured using the pNPG assay (77 pNPG U/mg). Table 1 lists the ratios of Trichoderma whole cellulase to BGL1 on a wt:wt basis, along with the corresponding activity units loaded per gram of cellulose in the substrate. Dividing the pNPG U/g value by the CMC U/g value yields a ratio of pNPG/CMC activity present in the mixture that is independent of substrate or enzyme loading.

TABLE 1

| wt:wt | | | |
|---|---|---|---|
| ratio of | | | |
| WC to | | | Ratio |
| BGL1 | CMC (U/g) | pNPG (U/g) | pNPG/CMC |
| 91:9 | 252 | 154 | 0.61 |
| 87:13 | 238 | 231 | 0.97 |
| 82:18 | 224 | 308 | 1.38 |
| 78:22 | 210 | 385 | 1.83 |
| 73:27 | 196 | 462 | 2.36 |
| 68:32 | 182 | 539 | 2.96 |
| 64:36 | 168 | 616 | 3.67 |
| 58:41 | 154 | 693 | 4.50 |
| 54:46 | 140 | 770 | 5.50 |
| 49:51 | 126 | 847 | 6.72 |
| 44:56 | 112 | 924 | 8.25 |
| 39:61 | 98 | 1001 | 10.21 |
| 33:37 | 84 | 1078 | 12.83 |
| 28:72 | 70 | 1155 | 16.50 |
| 23:77 | 56 | 1232 | 22.00 |

What is claimed is:

1. A method of decreasing the amount of a whole cellulase preparation required to hydrolyze a cellulosic material by adding an effective amount beta-glucosidase to form a beta-glucosidase enhanced whole cellulase, wherein the amount of said whole cellulase preparation required to hydrolyze said cellulosic material is decreased.

2. The method of claim 1 wherein the beta-glucosidase enhanced whole cellulase has about equal or greater specific performance specific performance relative to said whole cellulase preparation alone.

3. The method of claim 1 wherein the amount of beta-glucosidase is greater than 10% of the amount of the whole cellulase on a weight:weight ratio.

4. The method of claim 1 wherein the ratio of beta-glucosidase activity to whole cellulase activity is greater than 0.61 pNPG/CMC units.

5. The method of claim 1 wherein the amount of beta-glucosidase is less than 80% of the amount of whole cellulase preparation on a weight:weight ratio.

6. The method of claim 1 wherein the ratio of beta-glucosidase activity to whole cellulase activity is less than 22 pNPG/CMC units.

7. The method of claim 1 wherein the amount of beta-glucosidase is approximately equal to amount of whole cellulase on a weight:weight ratio.

8. The method of claim 1 wherein the ratio of beta-glucosidase activity to whole cellulase activity is about 5.5 pNPG/CMC units.

9. The method of claim 1 wherein said whole cellulase preparation comprises one or more cellobiohydrolases and endoglucanases.

10. The method of claim 1 where the beta-glucosidase decreases the amount of whole cellulase required to hydrolyze over 30% of the cellulosic material.

11. The method of claim 1 where the beta-glucosidase decreases the amount of whole cellulase required to hydrolyze over 30% of the cellulosic material, in about 48 hrs at 50° C.

12. The method of claim 1 wherein the whole cellulase preparation is a Trichoderma whole cellulase.

13. The method of claim 12 wherein said Trichoderma whole cellulase is a whole broth formulation.

14. The method of claim 1 wherein the beta-glucosidase is a Trichoderma beta-glucosidase.

15. The method of claim 14 wherein the Trichoderma beta-glucosidase is selected from BGL1, BGL3, BGL4, BGL5, BGL6 and BGL7 and any combination thereof.

16. A method of hydrolyzing a cellulosic material comprising:
contacting a cellulosic material with an effective amount a beta-glucosidase enhanced whole cellulase comprising about equal or greater specific performance relative to a whole cellulase alone.

17. The method of claim 16 wherein the beta-glucosidase enhanced whole cellulase comprises greater than 10% of beta-glucosidase relative to the whole cellulase on a weight:weight ratio.

18. The method of claim 16 wherein the beta-glucosidase enhanced whole cellulase comprises a ratio of beta-glucosidase activity to whole cellulase activity greater than 0.61 pNPG/CMC units.

19. The method of claim 16 wherein the beta-glucosidase enhanced whole cellulase comprises less than 80% of beta-glucosidase relative the whole cellulase on a weight:weight ratio.

20. The method of claim 16 wherein the beta-glucosidase enhanced whole cellulase comprises ratio of beta-glucosidase activity to whole cellulase activity greater than 0.61 pNPG/CMC units.

21. The method of claim 16 wherein the beta-glucosidase enhanced whole cellulase comprises a ratio of beta-glucosidase activity to whole cellulase activity of about less than 22 pNPG/CMC units.

22. The method of claim 16 wherein the beta-glucosidase enhanced whole cellulase comprises an amount of beta-glucosidase approximately equal to the amount of whole cellulase on a weight:weight ratio.

23. The method of claim 16 wherein the beta-glucosidase enhanced whole cellulase comprises a ratio of beta-glucosidase activity to whole cellulase activity of about 5.5 pNPG/CMC units.

24. The method of claim 16 wherein the beta-glucosidase enhanced whole cellulase comprises one or more cellobiohydrolases and endoglucanases.

25. The method of claim 16 where the beta-glucosidase enhanced whole cellulase comprises an amount of beta-glucosidase to decreases the amount of whole cellulase required to hydrolyze over 30% of the cellulosic material.

26. The method of claim 16 wherein the beta-glucosidase enhanced whole cellulase comprises an amount of beta-glucosidase to decreases the amount of whole cellulase required to hydrolyze over 30% of the cellulosic material, in about 48 hrs at 50° C.

27. The method of claim 16 wherein the wherein the beta-glucosidase enhanced whole cellulase comprises a Trichoderma whole cellulase.

28. The method of claim 16 wherein said Trichoderma whole cellulase is a whole broth formulation.

29. The method of claim 16 wherein the beta-glucosidase enhanced whole cellulase comprises a Trichoderma beta-glucosidase.

30. The method of claim 16 wherein the beta-glucosidase enhanced whole cellulase comprises a Trichoderma beta-glucosidase selected from BG11, BGL3, BGL4, BGL5, BGL6 and BGL7.

31. A beta-glucosidase enhanced whole cellulase comprising greater than 10% beta-glucosidase.

32. A beta-glucosidase enhanced whole cellulase comprising a ratio of beta-glucosidase activity to whole cellulase activity greater than 0.61 pNPG/CMC units

33. A beta-glucosidase enhanced whole cellulase comprising an amount of beta-glucosidase greater than 10% of the amount of the whole cellulase on a weight:weight ratio.

34. A beta-glucosidase enhanced whole cellulase comprising an effective amount of beta-glucosidase wherein said beta-glucosidase enhanced whole cellulase has about equal or greater specific performance relative to a whole cellulase preparation.

35. The beta-glucosidase enhanced whole cellulase of claim 34 wherein the amount of beta-glucosidase is less than 80% of the amount of whole cellulase on a weight:weight ratio.

36. The beta-glucosidase enhanced whole cellulase of claim 34 wherein the amount of beta-glucosidase is approximately equal the amount of whole cellulase on a weight:weight ratio.

37. The beta-glucosidase enhanced whole cellulase of claim 34, wherein the ratio of beta-glucosidase activity to whole cellulase activity is greater than 0.61 pNPG/CMC units.

38. The beta-glucosidase enhanced whole cellulase of claim 34, wherein the ratio of beta-glucosidase activity to whole cellulase activity is less than 22 pNPG/CMC units.

39. The beta-glucosidase enhanced whole cellulase of claim 34, wherein the ratio of beta-glucosidase activity to whole cellulase activity is about 5.5 pNPG/CMC units.

40. The beta-glucosidase enhanced whole cellulase of claim 34 wherein said whole cellulase preparation comprises one or more cellobiohydrolases and endoglucanases.

41. The beta-glucosidase enhanced whole cellulase of claim 34 wherein the whole cellulase preparation is a Trichoderma whole cellulase.

42. The beta-glucosidase enhanced whole cellulase of claim 34 wherein said Trichoderma whole cellulase is a whole broth formulation.

43. The beta-glucosidase enhanced whole cellulase of claim 34 wherein the beta-glucosidase is a Trichoderma beta-glucosidase.

44. The beta-glucosidase enhanced whole cellulase of claim 43 wherein the Trichoderma beta-glucosidase is selected from BGL1, BGL3, BGL4, BGL5, BGL6 and BGL7.
